(19) United States
(12) Patent Application Publication
(10) Pub. No.: US 2023/0382862 A1
(43) Pub. Date: Nov. 30, 2023

(54) ERG ONCOGENE INHIBITORS

(71) Applicants: Sanjay Malhotra, Bethesda, MD (US); Mallesh Pandrala, Bethesda, MD (US); The Henry M. Jackson Foundation for the Advancement of Military Medicine, Inc., Bethesda, MD (US)

(72) Inventors: Albert L. Dobi, Bethesda, MD (US); Sanjay Malhotra, Palo Alto, CA (US); Mallesh Pandrala, Palo Alto, CA (US)

(21) Appl. No.: 18/010,913

(22) PCT Filed: Jun. 18, 2021
(86) PCT No.: PCT/US2021/038051

§ 371 Date: Dec. 16, 2022

Related U.S. Application Data
(60) Provisional application No. 63/041,452, filed on Jun. 19, 2020.

(51) Int. Cl. C07D 213/26; C07C 309/04; A61P 35/00

(52) U.S. Cl. CPC C07D 213/26; C07C 309/04; A61P 35/00

(57) ABSTRACT

The present disclosure relates generally to compounds suitable as ERG inhibitors, including compositions comprising such compounds, methods for their use in treating diseases associated with overexpression of wild type ERG protein, an altered ERG protein, ERG gene transcription or ERG mRNA translation, and methods of making such compounds.

### CROSS-REFERENCE TO RELATED APPLICATIONS

This application claims priority to U.S. Provisional Application No. 63/041,452, filed Jun. 19, 2020, which is incorporated herein by reference for all purposes.

### STATEMENT REGARDING FEDERALLY SPONSORED RESEARCH OR DEVELOPMENT

This disclosure was made with government support under grant number HU0001-10-2-0002 awarded by the Uniformed Services University of the Health Sciences. The government has certain rights in the invention.

### FIELD

The present disclosure relates generally to compounds and compositions suitable as inhibitors of ERG. Also provided herein are methods of synthesizing such compounds.

### BACKGROUND

The ETS Related Gene (ERG) proto-oncogene was characterized more than thirty years ago and belongs to a large family of ETS transcription factors that regulate gene expression. These transcription factors are downstream effectors of the mitogenic signal transduction pathways involved in cell proliferation, cell differentiation, development, transformation, apoptosis, and immune regulation.

The cancer of prostate (CaP) is the most frequently diagnosed non-skin malignancy and second leading cause of cancer related deaths among men in the western countries. While early detected CaP due to PSA screening is managed effectively by surgery or radiation, a significant subset of CaP patients (20% to 40%) experience disease recurrence after definitive treatment and will require hormone ablation therapy. Despite an initial response to therapy, metastatic CaP eventually become refractory to hormone ablation therapy. For this subset of patients—i.e., those having metastatic hormone refractory cancer—there is currently no definitive cure.

The ERG gene is the most prevalent and validated genomic alteration in prostate cancer. The ERG proto-oncogene is overexpressed in 60-70% of prostate tumors in patients of Caucasian ancestry as a result of recurrent gene fusions involving TMPRSS2 and the ETS family of genes. Emerging studies on human prostate cancer specimens and various experimental models underscore the causative oncogenic function of ERG in prostate cancer. ETS factors reprogram the androgen receptor cistrome and prime prostate tumorigenesis through MYC oncogene or in response to PTEN loss. Numerous reports have highlighted both diagnostic and prognostic features of the genomic activation of ERG revealing that about half the prostate tumors harbor the most common gene fusion that takes place between the androgen receptor-regulated TMPRSS2 gene promoter and ERG protein coding sequence. Fusion between the TMPRSS2 gene promoter and ERG results in the overexpression of N-terminally truncated form of ERG.

ERG expression in CaP is androgen receptor (AR) dependent. While AR signaling inhibitors are employed as therapeutics for treating CaP, compounds that selectively inhibit ERG expression are highly desirable. An estimate of up to 4 million of patients living with prostate cancer worldwide harbor ERG positive tumors.

### SUMMARY

Provided herein is a pharmaceutically acceptable salt of Compound I:

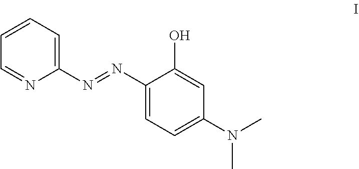 [SMILES: CN(C)c1ccc(/N=N/c2ccccn2)c(O)c1]

wherein the pharmaceutically acceptable salt is dihydroxybenzoic acid salt, hydrochloric acid salt, maleic acid salt, benzenesulfonic acid salt, or methanesulfonic acid salt.

Also provided herein is a methanesulfonic acid salt of Compound I.

Some embodiments provide for a composition comprising Compound I, wherein at least 97% of Compound I is a methanesulfonic acid salt.

Some embodiments provide for a pharmaceutical composition comprising Compound I, and a pharmaceutically acceptable excipient, wherein at least 97% of Compound I is a methanesulfonic acid salt.

Some embodiments provide for a pharmaceutical composition comprising Compound I, or a pharmaceutically acceptable salt thereof, and a pharmaceutically acceptable excipient, wherein the pharmaceutical composition is substantially free of solvent.

Some embodiments provide for methods for preparing a pharmaceutically acceptable salt of Compound I, wherein the pharmaceutically acceptable salt is dihydroxybenzoic acid salt, hydrochloric acid salt, maleic acid salt, benzenesulfonic acid salt, or methanesulfonic acid salt, comprising contacting Compound I with an acid selected from dihydroxybenzoic acid, hydrochloric acid, maleic acid, benzenesulfonic acid, or methanesulfonic acid under conditions sufficient to form the pharmaceutically acceptable salt of Compound I.

Some embodiments provide for methods for preparing a pharmaceutically acceptable salt of Compound I, wherein the pharmaceutically acceptable salt is dihydroxybenzoic acid salt, hydrochloric acid salt, maleic acid salt, benzenesulfonic acid salt, or methanesulfonic acid salt, comprising:
(a) contacting a Compound A:

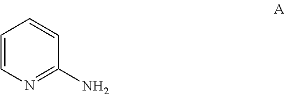

with an oxidizing agent under conditions sufficient to form Compound B:

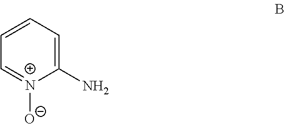

(b) contacting Compound B in the presence of sodium nitrite and a strong acid under conditions sufficient to form a salt of Compound C:

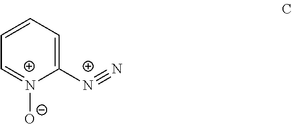

(c) contacting the salt of Compound C with Compound D:

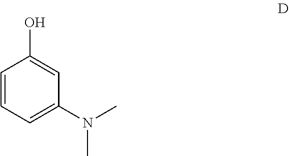

under conditions sufficient to form Compound E:

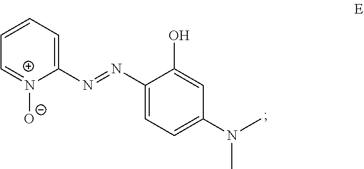 [SMILES: CN(C)c1ccc(/N=N/c2cccc[n+]2[O-])c(O)c1]

(d) contacting Compound E with a molybdenum catalyst and triphenylphosphine under conditions sufficient to form Compound I; and
(e) contacting Compound I with an acid selected from dihydroxybenzoic acid, hydrochloric acid, maleic acid, benzenesulfonic acid, or methanesulfonic acid under conditions sufficient to form the pharmaceutically acceptable salt of Compound I.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In other embodiments, the term “about” includes the indicated amount ±5%. In certain other embodiments, the term “about” includes the indicated amount ±1%. In certain other embodiments, the term “about” includes the indicated amount ±0.5%. In certain other embodiments, the term “about” includes the indicated amount ±0.2%. Also, to the term “about X” includes description of “X”.

Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to a salt that retains the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

A “pharmaceutically acceptable excipient” as used herein is non-toxic, aids administration, and does not adversely affect the therapeutic benefit of the compound described herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” or “patient” refers to an animal, such as a mammal (including a human), that has been or will be the object of treatment, observation or experiment. The methods described herein may be useful in human therapy and/or veterinary applications. In some embodiments, the subject is a mammal. In some embodiments, the subject is a human.

The term “therapeutically effective amount” or “effective amount” of a compound described herein or a pharmaceutically acceptable salt thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of sickle cell disease. The therapeutically effective amount may vary depending on the subject, and disease or condition being treated, the weight and age of the subject, the severity of the disease or condition, and the manner of administering, which can readily be determined by one or ordinary skill in the art. A therapeutically effective amount of a compound can be administered in one of more doses.

The term “administration” refers to introducing an agent into a subject (i.e. a patient). A therapeutic amount can be administered, which can be determined by the treating physician or the like. The related terms and phrases, “administering” and “administration of,” when used in connection with a compound or composition (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” refers to the total amount of active material (e.g., a compound as disclosed herein or a pharmaceutically acceptable salt thereof) administered to a patient at one time. The desired dose may be administered once daily.

The term “dosage” refers to total amount of active material (e.g. a compound as disclosed herein or pharmaceutically acceptable salt thereof) administered to a patient during a period of time, e.g. 24-hour period or daily. The desired dosage may be administered in one, two, three, four or more sub-doses at appropriate intervals throughout the the period, where the cumulative amount of the sub-doses equals the amount of the desired dose administered in a period.

The term “reaction conditions” is intended to refer to the physical and/or environmental conditions under which a chemical reaction proceeds.

The term “under conditions sufficient to” or “under reaction conditions sufficient to” is intended to refer to the reaction conditions under which the desired chemical reaction may proceed. Examples of reaction conditions include, but are not limited to, one or more of following: reaction temperature, solvent, pH, pressure, reaction time, mole ratio of reactants, the presence of a base or acid, or catalyst, radiation, concentration, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or may be readily obtained from the literature. Exemplary reaction conditions sufficient for performing the chemical transformations provided herein may be found throughout, and in particular, the examples below. It is also contemplated that the reaction conditions may include reagents in addition to those listed in the specific reaction.

The term “reagent” refers to a substance or compound that may be added to bring about a chemical reaction.

The term “solvent” refers to a substance inert under the conditions of the reaction being described in conjunction therewith.

The term “oxidizing agent” refers to a substance that removes electrons from other reactants (thereby gaining electrons itself). Non-limiting examples of an oxidizing agent include hydrogen peroxide, mCPBA, ozone, potassium permanganate, peroxides, and the like.

The term “strong acid” refers to an acid that is completely or nearly 100% ionized in solution. Non-limiting examples of a strong acid are hydrochloric acid, hydrobromic acid, perchloric acid, sulfuric acid, nitric acid, chloric acid, tetrafluoroboric acid, and the like.

The term “substantially free” when qualifying a component of a composition described herein is intended to mean that no more than 0.001%; no more than 0.01%; no more than 0.1%; no more than 0.5%; no more than 1%; no more than 5%; no more than 10%; or no more than 15% of the component is present in the composition.

### Compounds and Methods of Making Thereof

Provided herein is a pharmaceutically acceptable salt of Compound I:

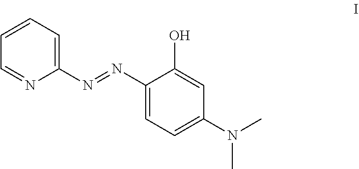 [SMILES: CN(C)c1ccc(/N=N/c2ccccn2)c(O)c1]

wherein the pharmaceutically acceptable salt is dihydroxybenzoic acid salt (i.e. gentisic acid salt), hydrochloric acid salt, maleic acid salt, benzenesulfonic acid salt, methanesulfonic acid salt, hydrobromic acid salt, sulfuric acid salt, sodium salt, nitric acid salt, tartaric acid salt, succinic acid salt, ethanedisulfonic acid salt, oxalic acid salt, ethanesulfonic acid salt, glycolic acid salt, toluenesulfonic acid salt, malic acid salt, naphthalene-2-sulfonic acid salt, naphthalene-1,5-disulfonic acid salt, or pyruvic acid salt.

Provided herein is a pharmaceutically acceptable salt of Compound I, wherein the pharmaceutically acceptable salt is dihydroxybenzoic acid salt (i.e. gentisic acid salt), hydrochloric acid salt, maleic acid salt, benzenesulfonic acid salt, methanesulfonic acid salt, hydrobromic acid salt, sulfuric acid salt, sodium salt, tartaric acid salt, succinic acid salt, ethanedisulfonic acid salt, or para-toluenesulfonic acid salt.

In some embodiments, the pharmaceutically acceptable salt is dihydroxybenzoic acid salt, hydrochloric acid salt, maleic acid salt, benzenesulfonic acid salt, or methanesulfonic acid salt.

In some embodiments, the pharmaceutically acceptable salt is dihydroxybenzoic acid salt. In some embodiments, the pharmaceutically acceptable salt is hydrochloric acid salt. In some embodiments, the pharmaceutically acceptable salt is maleic acid salt. In some embodiments, the pharmaceutically acceptable salt is benzenesulfonic acid salt. In some embodiments, the pharmaceutically acceptable salt is methanesulfonic acid salt.

Some embodiments provide for a methanesulfonic acid salt of Compound I.

Compound I, also known as (E)-5-(dimethylamino)-2-(pyridin-2-yldiazenyl)phenol, is selective for inhibiting the growth of ERG positive cancer cells and inhibiting ERG protein through direct binding to the RIOK2 atypical kinase, a putative upstream regulator of ERG.

Also provided herein are methods for preparing a pharmaceutically acceptable salt of Compound I. It is contemplated that such methods achieve high purity of Compound I or a pharmaceutically acceptable salt thereof.

Some embodiments provide for a composition comprising Compound I, wherein at least 95% of Compound I is a pharmaceutically acceptable salt. Some embodiments provide for a composition comprising Compound I, wherein at least 96% of Compound I is a pharmaceutically acceptable salt. Some embodiments provide for a composition comprising Compound I, wherein at least 97% of Compound I is a pharmaceutically acceptable salt. Some embodiments provide for a composition comprising Compound I, wherein at least 98% of Compound I is a pharmaceutically acceptable salt. Some embodiments provide for a composition comprising Compound I, wherein at least 99% of Compound I is a pharmaceutically acceptable salt.

In some embodiments, the pharmaceutically acceptable salt is dihydroxybenzoic acid salt (i.e. gentisic acid salt), hydrochloric acid salt, maleic acid salt, benzenesulfonic acid salt, methanesulfonic acid salt, hydrobromic acid salt, sulfuric acid salt, sodium salt, nitric acid salt, tartaric acid salt, succinic acid salt, ethanedisulfonic acid salt, oxalic acid salt, ethanesulfonic acid salt, glycolic acid salt, toluenesulfonic acid salt, malic acid salt, naphthalene-2-sulfonic acid salt, naphthalene-1,5-disulfonic acid salt, or pyruvic acid salt.

In some embodiments, the pharmaceutically acceptable salt is dihydroxybenzoic acid salt (i.e. gentisic acid salt), hydrochloric acid salt, maleic acid salt, benzenesulfonic acid salt, methanesulfonic acid salt, hydrobromic acid salt, sulfuric acid salt, sodium salt, tartaric acid salt, succinic acid salt, ethanedisulfonic acid salt, or para-toluenesulfonic acid salt.

Some embodiments provide for a composition comprising Compound I, wherein at least 95% of Compound I is a pharmaceutically acceptable salt, wherein the pharmaceutically acceptable salt is dihydroxybenzoic acid salt, hydrochloric acid salt, maleic acid salt, benzenesulfonic acid salt, or methanesulfonic acid salt. Some embodiments provide for a composition comprising Compound I, wherein at least 96% of Compound I is a pharmaceutically acceptable salt, wherein the pharmaceutically acceptable salt is dihydroxybenzoic acid salt, hydrochloric acid salt, maleic acid salt, benzenesulfonic acid salt, or methanesulfonic acid salt. Some embodiments provide for a composition comprising Compound I, wherein at least 97% of Compound I is a pharmaceutically acceptable salt, wherein the pharmaceutically acceptable salt is dihydroxybenzoic acid salt, hydrochloric acid salt, maleic acid salt, benzenesulfonic acid salt, or methanesulfonic acid salt. Some embodiments provide for a composition comprising Compound I, wherein at least 98% of Compound I is a pharmaceutically acceptable salt, wherein the pharmaceutically acceptable salt is dihydroxybenzoic acid salt, hydrochloric acid salt, maleic acid salt, benzenesulfonic acid salt, or methanesulfonic acid salt. Some embodiments provide for a composition comprising Compound I, wherein at least 99% of Compound I is a pharmaceutically acceptable salt, wherein the pharmaceutically acceptable salt is dihydroxybenzoic acid salt, hydrochloric acid salt, maleic acid salt, benzenesulfonic acid salt, or methanesulfonic acid salt.

Some embodiments provide for composition comprising Compound I, wherein at least 95% of Compound I is a methanesulfonic acid salt. Some embodiments provide for composition comprising Compound I, wherein at least 96% of Compound I is a methanesulfonic acid salt. Some embodiments provide for composition comprising Compound I, wherein at least 97% of Compound I is a methanesulfonic acid salt.

In some embodiments, at least 98% of Compound I is a methanesulfonic acid salt. In some embodiments, at least 99% of Compound I is a methanesulfonic acid salt.

In some embodiments, wherein the compositions described herein or pharmaceutical compositions described herein comprise an amount of Compound I as a pharmaceutically acceptable salt or as a methanesulfonic acid salt, the remaining amount of Compound I is Compound I, or a pharmaceutically acceptable salt thereof, or a mixture thereof.

Some embodiments provide for a composition comprising Compound I, or a pharmaceutically acceptable salt thereof, wherein the composition is substantially free of impurities.

Some embodiments provide for a composition comprising Compound I, or a pharmaceutically acceptable salt thereof, wherein the composition is substantially free of solvent.

Some embodiments provide for methods for preparing a pharmaceutically acceptable salt of Compound I, wherein the pharmaceutically acceptable salt is dihydroxybenzoic acid salt (i.e. gentisic acid salt), hydrochloric acid salt, maleic acid salt, benzenesulfonic acid salt, methanesulfonic acid salt, hydrobromic acid salt, sulfuric acid salt, sodium salt, nitric acid salt, tartaric acid salt, succinic acid salt, ethanedisulfonic acid salt, oxalic acid salt, ethanesulfonic acid salt, glycolic acid salt, toluenesulfonic acid salt, malic acid salt, naphthalene-2-sulfonic acid salt, naphthalene-1,5-disulfonic acid salt, or pyruvic acid salt, comprising:
contacting Compound I with dihydroxybenzoic acid, hydrochloric acid, maleic acid, benzenesulfonic acid, methanesulfonic acid, hydrobromic acid, sulfuric acid, Na (such as sodium hydroxide), nitric acid, tartaric acid, succinic acid, ethanedisulfonic acid, oxalic acid, ethanesulfonic acid, glycolic acid, toluenesulfonic acid, malic acid, naphthalene-2-sulfonic acid, naphthalene-1,5-disulfonic acid, or pyruvic acid, under conditions sufficient to form the pharmaceutically acceptable salt of Compound I.

Some embodiments provide for methods for preparing a pharmaceutically acceptable salt of Compound I, wherein the pharmaceutically acceptable salt is dihydroxybenzoic acid salt (i.e. gentisic acid salt), hydrochloric acid salt, maleic acid salt, benzenesulfonic acid salt, methanesulfonic acid salt, hydrobromic acid salt, sulfuric acid salt, sodium salt, nitric acid salt, tartaric acid salt, succinic acid salt, ethanedisulfonic acid salt, oxalic acid salt, ethanesulfonic acid salt, glycolic acid salt, toluenesulfonic acid salt, malic acid salt, naphthalene-2-sulfonic acid salt, naphthalene-1,5-disulfonic acid salt, or pyruvic acid salt, comprising:
contacting a salt of Compound I with dihydroxybenzoic acid, hydrochloric acid, maleic acid, benzenesulfonic acid, methanesulfonic acid, hydrobromic acid, sulfuric acid, Na (such as sodium hydroxide), nitric acid, tartaric acid, succinic acid, ethanedisulfonic acid, oxalic acid, ethanesulfonic acid, glycolic acid, toluenesulfonic acid, malic acid, naphthalene-2-sulfonic acid, naphthalene-1,5-disulfonic acid, or pyruvic acid, under conditions sufficient to form the pharmaceutically acceptable salt of Compound I.

Some embodiments provide for methods for preparing a pharmaceutically acceptable salt of Compound I, wherein the pharmaceutically acceptable salt is dihydroxybenzoic acid salt, hydrochloric acid salt, maleic acid salt, benzenesulfonic acid salt, or methanesulfonic acid salt, comprising contacting Compound I with an acid selected from dihydroxybenzoic acid, hydrochloric acid, maleic acid, benzenesulfonic acid, or methanesulfonic acid under conditions sufficient to form the pharmaceutically acceptable salt of Compound I.

Some embodiments provide for methods for preparing a pharmaceutically acceptable salt of Compound I, wherein the pharmaceutically acceptable salt is dihydroxybenzoic acid salt, hydrochloric acid salt, maleic acid salt, benzenesulfonic acid salt, or methanesulfonic acid salt, comprising:
(a) contacting a Compound A:

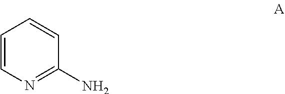

with an oxidizing agent under conditions sufficient to form Compound B:

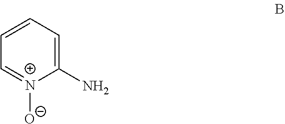

(b) contacting Compound B in the presence of sodium nitrite and a strong acid under conditions sufficient to form a salt of Compound C:

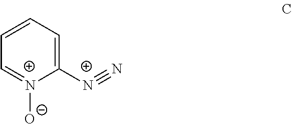

(c) contacting the salt of Compound C with Compound D:

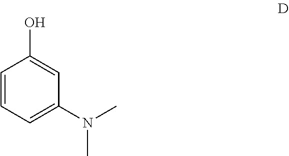

under conditions sufficient to form Compound E:

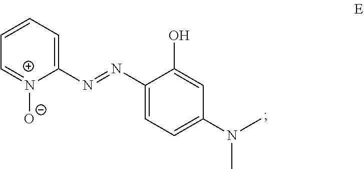 [SMILES: CN(C)c1ccc(/N=N/c2cccc[n+]2[O-])c(O)c1]

(d) contacting Compound E with a molybdenum catalyst and triphenylphosphine under conditions sufficient to form Compound I; and
(e) contacting Compound I with an acid selected from dihydroxybenzoic acid, hydrochloric acid, maleic acid, benzenesulfonic acid, or methanesulfonic acid under conditions sufficient to form the pharmaceutically acceptable salt of Compound I.

In some embodiments, the reaction conditions of step (a) comprise a temperature of about 15 and 30° C. In some embodiments, the reaction conditions of step (a) comprise a temperature of about 20 and 25° C. In some embodiments, the oxidizing agent is meta-chloroperoxybenzoic acid.

In some embodiments, the reaction conditions of step (b) comprise a temperature of about −20 and 0° C. In some embodiments, the reaction conditions of step (b) comprise a temperature of about −15 and 0° C. In some embodiments, the strong acid is tetrafluoroboric acid.

In some embodiments, the salt of Compound C is a tetrafluoroboric acid salt.

In some embodiments, the reaction conditions of step (c) comprise a temperature of about 5 and 30° C. In some embodiments, the reaction conditions of step (c) comprise a temperature of about 15 and 25° C.

In some embodiments, the reaction conditions of step (d) comprise a temperature of about 15 and 30° C. In some embodiments, the reaction conditions of step (d) comprise a temperature of about 20 and 25° C. In some embodiments, the molybdenum catalyst is MoO₂[(CH₂H₅)₂NCS₂]₂.

In some embodiments, the acid is methanesulfonic acid. In such embodiments, the pharmaceutically acceptable salt of Compound I is methanesulfonic acid salt.

### Pharmaceutical Compositions

Some embodiments provide for a pharmaceutical composition comprising a pharmaceutically acceptable salt of Compound I as described herein and a pharmaceutically acceptable excipient.

Some embodiments provide for a pharmaceutical composition comprising Compound I, and a pharmaceutically acceptable excipient, wherein at least 97% of Compound I is a methanesulfonic acid salt.

In some embodiments, at least 98% of Compound I is a methanesulfonic acid salt. In some embodiments, at least 99% of Compound I is a methanesulfonic acid salt.

Some embodiments provide for a pharmaceutical composition comprising Compound I, and a pharmaceutically acceptable excipient, wherein the pharmaceutical composition is substantially free of impurities.

Some embodiments provide for a pharmaceutical composition comprising Compound I, and a pharmaceutically acceptable excipient, wherein the pharmaceutical composition is substantially free of solvent.

In some embodiments, the pharmaceutical compositions provided herein comprise one or more pharmaceutically acceptable vehicles selected from carriers, adjuvants and excipients.

Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

In some embodiments, the pharmaceutical compositions described herein are suitable for oral delivery. In some embodiments, the pharmaceutical compositions described herein are administered intravenously.

Oral administration may be via, for example, capsule or tablet, which may be optionally enterically coated. In making the pharmaceutical compositions that include a compound described herein, or a pharmaceutically acceptable salt thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include a compound described herein, or a pharmaceutically acceptable salt thereof, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Treatment Methods and Uses

Provided herein are methods for treating a disease associated with overexpression of wild type ERG protein, an altered ERG protein, ERG gene transcription or ERG mRNA translation in a subject in need thereof, comprising administering a therapeutically effective amount of a pharmaceutically acceptable salt of a compound as described herein, or a composition as described herein, or a pharmaceutical composition as described herein.

In some embodiments, the disease is a cancer. In some embodiments, the disease is prostate cancer, colorectal cancer, Ewing sarcoma, or leukemia. In some embodiments, the disease is prostate cancer or colorectal cancer. In some embodiments, the disease is prostate cancer. In some embodiments, the disease is colorectal cancer.

Some embodiments provide for methods of treating a disease associated with overexpression of wild type ERG protein, an altered ERG protein, ERG gene transcription or ERG mRNA translation in a subject in need thereof, comprising administering a therapeutically effective amount of a pharmaceutically acceptable salt of a compound as described herein, a composition as described herein, or a pharmaceutical composition as described herein, wherein the disease is prostate cancer, colorectal cancer, Ewing sarcoma, or leukemia.

Provided herein are methods for treating a cancer in a subject in need thereof, comprising administering a therapeutically effective amount of a pharmaceutically acceptable salt of a compound as described herein, or a composition as described herein, or a pharmaceutical composition as described herein.

In some embodiments, the cancer is prostate cancer, colorectal cancer, Ewing sarcoma, or leukemia.

In some embodiments, the leukemia is acute lymphocytic leukemia, acute myeloid leukemia, chronic lymphcytic leukemia, or chornic myeloid leukemia.

Provided herein are methods for treating an ERG-positive cancer in a subject in need thereof, comprising administering a therapeutically effective amount of a pharmaceutically acceptable salt of a compound as described herein, or a composition as described herein, or a pharmaceutical composition as described herein.

Provided herein are methods for treating prostate cancer in a subject in need thereof, comprising administering a therapeutically effective amount of a pharmaceutically acceptable salt of a pharmaceutically acceptable salt of a compound as described herein, or a composition as described herein, or a pharmaceutical composition as described herein.

Provided herein are methods of treating ERG-positive prostate cancer, comprising administering a therapeutically effective amount of a pharmaceutically acceptable salt of a compound as described herein, a composition as described herein, or a pharmaceutical composition as described herein.

In some embodiments, a compound, or a pharmaceutically acceptable salt thereof, as described herein are administered as a first line therapy. In some embodiments, a compound, or a pharmaceutically acceptable salt thereof, as described herein are administered as a second line therapy or a third line therapy. In some embodiments, a compound, or a pharmaceutically acceptable salt thereof, as described herein are administered subsequent to a third line therapy.

As used herein, a first line therapy is the therapeutic regimen that is first prescribed or followed upon diagnosis of a condition that warrants the use of an ERG inhibitor, such as but not limited to prostate cancer. A second line therapy is the therapeutic regimen that is prescribed or followed upon diagnosis of a recurrence or metastasis of condition that warrants the use of an ERG inhibitor, such as but not limited to prostate cancer. Likewise, a third line therapy is the therapeutic regimen that is prescribed or followed upon diagnosis of a second recurrence or metastasis of condition that warrants the use of an ERG inhibitor, such as but not limited to prostate cancer. A therapy, for the purposes of determining which “line” of therapy as used herein, need not be a drug therapy. For example, a first line therapy, as used herein, may be surgical removal, or radiation therapy. Any therapy designed to remove, reduce or ablate the tumor or condition can be considered a “line” of therapy.

In some embodiments, a compound, or a pharmaceutically acceptable salt thereof, as described herein can be administered as a “maintenance” therapeutic. As used herein, a maintenance therapeutic is a therapeutic regimen that is prescribed or followed while the subject is free of any detectable condition requiring treatment, for example, after a tumor is surgically removed from the subject. In some embodiments, a compound, or a pharmaceutically acceptable salt thereof, as described herein can be taken, for example, after surgical resection, for a specified period of time (such as, but not limited to, at least about six months, such as one year, two years, three years, four years or five years), or after the removal or disappearance of the tumor or cancer.

Dose

Typical dosage levels for a compound, or a pharmaceutically acceptable salt thereof, as described herein generally range from about 0.001 to about 100 mg per kg of the subject's body weight per day which can be administered in single or multiple doses. An exemplary dosage is about 0.01 to about 25 mg/kg per day or about 0.05 to about 10 mg/kg per day. In some embodiments, the dosage level ranges from about 0.01 to about 25 mg/kg per day, such as about 0.05 to about 10 mg/kg per day, or about 0.1 to about 5 mg/kg per day.

A dose can typically range from about 0.1 mg to about 2000 mg per day and can be given as a single once-a-day dose or, alternatively, as divided doses throughout the day, optionally taken with food. In some embodiments, the daily dose is administered twice daily in equally divided doses. A daily dose range can range from about 5 mg to about 500 mg per day such as, for example, between about 10 mg and about 300 mg per day. In managing the patient, the therapy can be initiated at a lower dose, such as from about 1 mg to about 25 mg, and increased if necessary up to from about 200 mg to about 2000 mg per day as either a single dose or divided doses, depending on the subject's global response.

### Combination Therapy

A compound, or a pharmaceutically acceptable salt thereof, described herein may be administered in combination with one or more additional active agents. Thus, in some embodiments, the methods described herein further comprise administering an additional active agent.

Some embodiments provide for methods for treating a disease associated with overexpression of wild type ERG protein, an altered ERG protein, ERG gene transcription or ERG mRNA translation in a subject in need thereof comprising administering to a subject, simultaneously or sequentially, a compound, or a pharmaceutically acceptable salt thereof, described herein and one or more additional active agent(s).

In methods using simultaneous administration, the compound, or a pharmaceutically acceptable salt thereof, described herein and the additional active agent(s) can be present in a combined composition or can be administered separately. When used in combination with one or more additional active agents, a compound described herein may be administered prior to, concurrently with, or following administration of the additional active agents. The administration can be by the same route or by different routes. In some embodiments, a compound, or a pharmaceutically acceptable salt thereof, described herein may be administered in combination with a second active agent.

In some embodiments, the additional active agent is an anti-cancer agent.

Also provided is a pharmaceutical composition comprising a compound, or a pharmaceutically acceptable salt thereof, described herein and an additional active agent.

In some embodiments, the additional active agent is a chemotherapeutic agent useful for treating an an ERG-positive cancer.

In some embodiments, a compound, or a pharmaceutically acceptable salt thereof, as described herein is administered in combination with a prostate cancer therapy. In some embodiments, a compound, or a pharmaceutically acceptable salt thereof, as described herein are administered in combination with one or more of lutenizing hormone-releasing hormone (LHRH) analogs such as, but not limited to, leuprolide (Lupron®, Eligard®), goserelin (Zoladex®), triptorelin (Trelstar®), degarelix (Firmagon®), Abiraterone (Zytiga®) and histrelin (Vantas®). In some embodiments, a compound, or a pharmaceutically acceptable salt thereof, as described herein are administered in combination with one or more of anti-androgen receptors such as, but not limited to, flutamide (Eulexin®), bicalutamide (Casodex®), Enzalutamide (Xtandi®), nilutamide (Nilandron®), and apalutamide (Erleada®). In some embodiments, a compound, or a pharmaceutically acceptable salt thereof, as described herein are administered in combination with one or more chemotherapeutics such as, but not limited to, Docetaxel (Taxotere®), Cabazitaxel (Jevtana®), Mitoxantrone (Novantrone®), Estramustine (Emcyt®), Doxorubicin (Adriamycin®), Etoposide (VP-16), Vinblastine (Velban®), Paclitaxel (Taxol®), Carboplatin (Paraplatin®), Vinorelbine (Navelbine®) Abiraterone (Zytiga), ARN-509 (J@J), and Galeterone (Tokai). In some embodiments, a compound, or a pharmaceutically acceptable salt thereof, as described herein are administered in combination with one or more of poly (ADP-ridobse) polymerase (“PARP”) inhibitor, such as, but not limited to, olaparib, niraparib, and rucaparib.). In some embodiments, a compound, or a pharmaceutically acceptable salt thereof, as described herein are administered in combination with one or more of a platinum-based chemotherapeutic, such as but not limited to, satraplatin, cisplatin, and carboplatin. In some embodiments, a compound, or a pharmaceutically acceptable salt thereof, as described herein are administered in combination with one or more of immunotherapeutic, such as, but not limited to, vaccines (such as but not limited to sipuleucel-T) and PD-1 inhibitors (such as but not limited to pembrolizumab). In some embodiments, a compound, or a pharmaceutically acceptable salt thereof, as described herein are administered in combination with one or more additional active agent as described herein.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

### Example 1: Synthesis of Compound I

Compound I was synthesized as shown in Scheme 1.

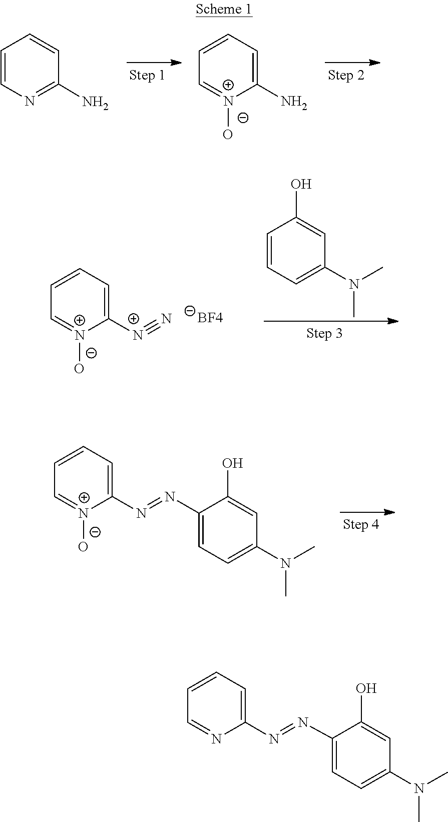

Step 1: mCPBA (70%, 2.88 g, 11.6 mmol) was dissolved in acetone (20 mL), dried over Na₂SO₄ for 30-40 min, and then added dropwise to a stirred solution of 2-aminopyridine in acetone (10 mL) at room temperature. The mixture was stirred at room temperature for 1 h and evaporated to dryness. The crude was purified with CombiFlash chromatography on silica gel using 0-10% of methanol in DCM as an eluent to afford 2-aminopyridine 1-oxide. 1H NMR (400 MHz, DMSO-d₆) δ 8.01 (dd, J=6.5, 1.5 Hz, 1H), 7.09 (ddd, J=8.6, 7.3, 1.5 Hz, 1H), 6.93-6.71 (m, 3H), 6.56 (td, J=6.9, 1.9 Hz, 1H). 13C NMR (101 MHz, DMSO-d₆) δ 151.17, 137.46, 127.32, 112.55, 109.47.

Step 2: 2-aminopyridine 1-oxide (0.8 g, 6.55 mmol) was taken in 4 mL HBF₄ and cooled to −5° C. to −10° C. To this solution, a solution of NaNO₂ (0.56 g, 8.11 mmol) in water (1.0 mL) was added slowly with stirring at such rate that the temperature was kept below −5° C. After the addition was complete, stirring was continued for 1 h at −5° C., during which time yellow precipitation formed. The hygroscopic yellow solid was collected carefully by decanting the clear solution under nitrogen atmosphere. It was washed thoroughly with minimal THF and followed by ether, and dried in vacuum at room temperature for 2 h to afford the desired compound.

Step 3: A stirred solution of 3-(dimethylamino)phenol (12.0 g, 87.4 mmol) in dry methanol (150 mL) under nitrogen atmosphere was cooled to 15° C. A solution of pyridine-2-diazonium 1-oxide tetrafluoroborate (20.0 g, 96.2 mmol) in methanol (250 mL) was added slowly over the course of 40 min, and the mixture stirred for 30-45 min at 15° C. The mixture was brought to room temperature and allowed to stir overnight, during which time a brown-red solid formed. The solid was collected by filtration and washed with copious amounts of hexane. The solid was taken in 150 mL of water, stirred for 30 min, and filtered and washed with hexane. The solid was dried in vacuum for 4 h at 60° C. to afford the desired compound.

Step 4: Triphenylphosphine (3.65 g, 13.5 mmol) and MoO₂[(C₂H₅)₂NCS₂]₂ (11.8 g, 28 mmol) was added to a stirred solution of (E)-2-((4-(dimethylamino)-2-hydroxyphenyl)diazenyl)pyridine 1-oxide (3.5 g, 13.5 mmol) in acetone (200 mL) at room temperature, and stirred for overnight. The unwanted solid formed during the reaction was removed by filtration. The filtrate was evaporated to dryness to afford the crude product, which was purified with CombiFlash chromatography on silica gel using 0-10% of methanol in DCM as an eluent. Combined pure fractions was evaporated to dryness to afford the desired product. The solid was recrystallized from isopropanol to give (E)-5-(dimethylamino)-2-(pyridin-2-yldiazenyl)phenol. ¹H NMR (400 MHz, Chloroform-d) δ 8.48 (ddd, J=4.9, 1.9, 0.9 Hz, 1H), 7.80-7.69 (m, 1H), 7.61 (dt, J=8.2, 1.0 Hz, 1H), 7.32 (d, J=9.6 Hz, 1H), 7.10 (ddq, J=7.3, 5.0, 1.3 Hz, 1H), 6.52-6.44 (m, 1H), 5.88 (d, J=2.8 Hz, 1H), 3.12 (s, 6H), 3.12 (d, J=2.6 Hz, 1H). ¹³C NMR (101 MHz, cdcl3) δ 169.19, 157.80, 156.28, 149.04, 138.05, 135.32, 133.05, 120.93, 112.03, 110.23, 99.13, 40.33. LC-MS (ESI-QQQ): m/z 243.1 ([C₁₃H₁₄N₄O+H]+ calcd. 243.1). Purity >99%. Elemental analysis: Anal. Cald. for C₁₃H₁₄N₄O: C, 64.45; H, 5.82; N, 23.13%. Found: C, 64.19, H, 5.95, N, 23.05%.

### Example 2: Synthesis of Salts of Compound I

Salts of Compound I were made according to either Procedure A or Procedure B and are summarized in Table 1.

Procedure A: Compound I (0.20 mmol) was taken in a clean and dry single-neck round-bottomed flask, and solvent (see Table 1) was added. The mixture was stirred for 5 to 10 min to observe clear solution. Then, counterion (“B” of Table 1, 2 eq, 0.40 mmol) was added slowly (if B is solid, it was dissolved in respective solvent, and then added, slowly). The mixture was stirred at room temperature for 18 h, and the resulting solid was collected by filtration. The solid was washed with copious amounts of respective solvent that was used for the reaction, and dried in vacuum at 60° C. for 4 h to afford the desired product.

Procedure B: Compound I (0.20 mmol) was taken in a clean and dry single-neck round-bottomed flask, and solvent (see Table 1) was added. The mixture was stirred for 5 to 10 min to observe clear solution. Then, counterion (“B” of Table 1, 2 eq, 0.40 mmol) was added slowly. The mixture was stirred at room temperature for 18 h. The solvent was evaporated either completely or partially until solid formation was observed; then, the solid was collected and washed with minimal amount of ether or hexane. The solid was dried in vacuum at 60° C. for 4 h to afford the desired product.

TABLE 1

| | | | | Reaction | Characterization |
|---|---|---|---|---|---|
| Name | # | B | Solvent | Procedure | Data |
| (E)-5- | 1 | HCl | 1,4-dioxane | A | ¹H NMR (400 |
| (dimethylamino)- | | | | | MHz, DMSO-d₆) |
| 2-(pyridin-2- | | | | | δ 8.52 (ddd, J = |
| yldiazenyl)phenol, | | | | | 5.7, 1.8, 0.8 Hz, |
| hydrochloride | | | | | 1H), 8.32 (t, J = |
| (1:2) | | | | | 8.0 Hz, 1H), 8.00 |
| | | | | | (d, J = 8.5 Hz, |
| | | | | | 1H), 7.60 (d, J = |
| | | | | | 9.5 Hz, 1H), 7.51 |
| | | | | | (t, J = 6.5 Hz, |
| | | | | | 1H), 6.79 (dd, J = |
| | | | | | 9.7, 2.7 Hz, 1H), |
| | | | | | 6.18 (d, J = 2.5 Hz, |
| | | | | | 1H), 3.23 (s, 6H). |
| | | | | | Elemental analysis: Anal. |
| | | | | | Cald. for {Compound |
| | | | | | I:HCl:Methanol |
| | | | | | (1:2:1)} |
| | | | | | C₁₄H₂₀Cl₂N₄O₂: |
| | | | | | C, 48.43; H, 5.81; |
| | | | | | N, 16.14%. |
| | | | | | found: C, 48.59; |
| | | | | | H, 5.37; N, 16.4%. |
| 5- | 2 | Methanesulfonic | Ethanol | A | ¹H NMR (400 |
| (dimethylamino)- | | acid | | | MHz, DMSO-d₆) |
| 2-(pyridin-2- | | | | | δ 8.53 (ddd, J = |
| yldiazenyl)phenol, | | | | | 5.6, 1.8, 0.8 Hz, |
| methanesulfonic | | | | | 1H), 8.23 (t, J = |
| acid (1:1) | | | | | 8.0 Hz, 1H), 7.95 |
| | | | | | (d, J = 8.5 Hz, |
| | | | | | 1H), 7.48 (dt, J = |
| | | | | | 11.8, 7.8 Hz, 2H), |
| | | | | | 6.80 (dt, J = 9.6, |
| | | | | | 2.9 Hz, 1H), 6.10 |
| | | | | | (d, J = 2.6 Hz, |
| | | | | | 1H), 3.22 (d, J = |
| | | | | | 3.2 Hz, 6H), 2.39- |
| | | | | | 2.28 (m, 3H). |
| | | | | | Elemental |
| | | | | | analysis: Anal. |
| | | | | | Cald. for {Compound |
| | | | | | I:methanesulfonic |
| | | | | | acid (1:1)} |
| | | | | | C₁₄H₁₈N₄O₄S: |
| | | | | | C, 49.69; H, 5.36, |
| | | | | | N: 16.56%. |
| | | | | | Found: C, 49.39; |
| | | | | | H, 5.28; N: 16.45%. |
| (E)-5- | 3 | Benzenesulfonic | Isopropanol | A | ¹H NMR (400 |
| (dimethylamino)- | | acid | | | MHz, DMSO-d₆) |
| 2-(pyridin-2- | | | | | δ 8.54 (dd, J = |
| yldiazenyl)phenol, | | | | | 5.6, 1.2 Hz, 1H), |
| benzenesulfonic | | | | | 8.25 (d, J = 7.8 |
| acid (1:1) | | | | | Hz, 1H), 7.98 (d, |
| | | | | | J = 8.3 Hz, 1H), |
| | | | | | 7.70-7.43 (m, |
| | | | | | 4H), 7.30 (dddd, J = |
| | | | | | 6.3, 4.3, 3.2, 2.2 |
| | | | | | Hz, 3H), 6.86- |
| | | | | | 6.75 (m, 1H), |
| | | | | | 6.13 (s, 1H), 3.24- |
| | | | | | 3.19 (m, 6H). |
| | | | | | Elemental |
| | | | | | analysis: Anal. |
| | | | | | Cald. for {Compound |
| | | | | | I:benzenesulfonic |
| | | | | | acid (1:1)} |
| | | | | | C₁₉H₂₀N₄O₄S: |
| | | | | | C, 56.99, H, 5.03, |
| | | | | | N, 13.99%. |
| | | | | | Found: C, 56.86; |
| | | | | | H, 4.96; N, 13.86%. |
| 5- | 4 | 2,5- | Methanol | A | ¹H NMR (400 |
| (dimethylamino)- | | dihydroxybenzoic | | | MHZ, DMSO-d₆) |
| 2-(pyridin-2- | | acid | | | δ 9.13 (s, 1H), |
| yldiazenyl)phenol, | | | | | 8.42 (ddd, J = 4.9, |
| 2,5- | | | | | 1.9, 0.9 Hz, 1H), |
| dihydroxybenzoic | | | | | 7.89 (ddd, J = 8.2, |
| acid (2:1) | | | | | 7.3, 1.8 Hz, 1H), |
| | | | | | 7.66 (dt, J = 8.2, |
| | | | | | 1.0 Hz, 1H), 7.28- |
| | | | | | 7.17 (m, 2H), |
| | | | | | 7.15 (dd, J = 3.1, |
| | | | | | 0.4 Hz, 1H), 6.95 |
| | | | | | (dd, J = 8.9, 3.1 |
| | | | | | Hz, 1H), 6.78 (d, |
| | | | | | J = 8.9 Hz, 1H), |
| | | | | | 6.75-6.68 (m, |
| | | | | | 1H), 5.78 (dd, J = |
| | | | | | 2.7, 1.3 Hz, 1H), |
| | | | | | 3.13 (d, J = 1.4 |
| | | | | | Hz, 6H). |
| | | | | | Elemental |
| | | | | | analysis: Anal. |
| | | | | | Cald. for {Compound |
| | | | | | I:2,5-dihydroxybenzoic |
| | | | | | acid (2:1)} |
| | | | | | C₃₃H₃₄N₈O₆: |
| | | | | | C, 62.06; H, 5.37; |
| | | | | | N, 17.54%. |
| | | | | | Found: C, 62.14; |
| | | | | | H, 5.26; N, 17.64%. |
| 5- | 5 | Maleic | Acetonitrile | A | ¹H NMR (400 |
| (dimethylamino)- | | acid | | | MHz, DMSO-d₆) |
| 2-(pyridin-2- | | | | | δ 8.43 (ddt, J = |
| yldiazenyl)phenol, | | | | | 5.0, 1.7, 0.8 Hz, |
| maleic acid (1:1) | | | | | 1H), 7.92 (ddd, J = |
| | | | | | 8.8, 7.3, 1.9 Hz, |
| | | | | | 1H), 7.69 (d, J = |
| | | | | | 8.3 Hz, 1H), 7.28- |
| | | | | | 7.20 (m, 2H), |
| | | | | | 6.73 (ddd, J = 9.7, |
| | | | | | 2.7, 0.6 Hz, 1H), |
| | | | | | 6.33-6.18 (m, |
| | | | | | 2H), 5.81 (d, J = |
| | | | | | 2.6 Hz, 1H), 3.26- |
| | | | | | 3.07 (m, 6H), |
| | | | | | 2.11-2.02 (m, |
| | | | | | 1H). Elemental |
| | | | | | analysis: Anal. |
| | | | | | Cald. for {Compound |
| | | | | | I:maleic acid (1:1)} |
| | | | | | C₁₇H₁₈N₄O₅: |
| | | | | | C, 56.98; H, 5.06; |
| | | | | | N, 15.63%. |
| | | | | | Found: C, 57.24 |
| | | | | | H, 4.95; N, 15.75%. |
| 5- | 6 | HBr | Ethyl | A | ¹H NMR (400 |
| (dimethylamino)- | | | acetate | | MHz, DMSO-d₆) |
| 2-(pyridin-2- | | | | | δ 8.63-8.52 (m, |
| yldiazenyl)phenol, | | | | | 1H), 8.43-8.31 |
| hydrogen bromide, | | | | | (m, 1H), 8.07 (d, |
| hydrate (1:2:0.5) | | | | | J = 8.6 Hz, 1H), |
| | | | | | 7.57 (ddd, J = 7.1, |
| | | | | | 5.8, 1.2 Hz, 2H), |
| | | | | | 6.84 (dd, J = 9.8, |
| | | | | | 2.6 Hz, 1H), 6.24 |
| | | | | | (d, J = 2.6 Hz, |
| | | | | | 1H), 3.26 (s, 6H). |
| | | | | | Elemental |
| | | | | | analysis: Anal. |
| | | | | | Cald. for {Compound |
| | | | | | I:hydrobromic |
| | | | | | acid:water (1:2:0.5)} |
| | | | | | C₁₃H₁₇Br₂N₄O_{1.5}: |
| | | | | | C, 37.80; H, 4.15; |
| | | | | | N, 13.56%. |
| | | | | | Found: C, 37.9, |
| | | | | | H, 4.24; N, 13.39%. |
| 5- | 7 | H₂SO₄ | Ethyl | A | ¹H NMR (400 |
| (dimethylamino)- | | | acetate | | MHz, DMSO-d₆) |
| 2-(pyridin-2- | | | | | δ 8.54 (d, J = 5.7 |
| yldiazenyl)phenol, | | | | | Hz, 1H), 8.31 (t, J = |
| sulfuric acid (1:2) | | | | | 7.9 Hz, 1H), |
| | | | | | 8.02 (d, J = 8.5 |
| | | | | | Hz, 1H), 7.61- |
| | | | | | 7.48 (m, 2H), |
| | | | | | 6.81 (dd, J = 9.7, |
| | | | | | 2.7 Hz, 1H), 6.20- |
| | | | | | 6.15 (m, 1H), |
| | | | | | 3.23 (s, 6H). |
| | | | | | Elemental |
| | | | | | analysis: Anal. |
| | | | | | Cald. for {Compound |
| | | | | | I:sulfuric |
| | | | | | acid (1:2)} |
| | | | | | C₁₃H₁₈N₄O₉S₂: |
| | | | | | C, 35.61, H, 4.14; |
| | | | | | N, 12.78%. |
| | | | | | Found: C, 35.80; |
| | | | | | H, 4.00; N, 12.57%. |
| Sodium-5- | 8 | Na⁺ | Water | A | ¹H NMR (400 |
| (dimethylamino)- | | | | | MHz, DMSO-d₆) |
| 2-(pyridin-2- | | | | | δ 8.30 (s, 1H), |
| yldiazenyl)phenolate, | | | | | 7.78 (s, 1H), 7.49 |
| Hydrate | | | | | (s, 1H), 7.32 (s, |
| | | | | | 1H), 7.08 (s, 1H), |
| | | | | | 5.58 (s, 1H), 2.99 |
| | | | | | (s, 6H). Elemental |
| | | | | | analysis: Anal. |
| | | | | | Cald. for |
| | | | | | {(sodium (E)-5- |
| | | | | | (dimethylamino)- |
| | | | | | 2-(pyridin-2- |
| | | | | | yldiazenyl)phenolate:water |
| | | | | | (1:0.8)} |
| | | | | | C₁₃H_{14.6}N₄NaO_{1.8}: |
| | | | | | C, 56.03; H, 5.28; |
| | | | | | N, 20.11%. |
| | | | | | Found: C, 56.01; |
| | | | | | H, 4.85; N, 19.44%. |
| 5- | 9 | HNO₃ | Ethanol | A | Hz, 1H), 8.20 (t, J = |
| (dimethylamino)- | | | | | 8.0 Hz, 1H), |
| 2-(pyridin-2- | | | | | 7.93 (d, J = 8.4 |
| yldiazenyl)phenol, | | | | | Hz, 1H), 7.45 (q, |
| nitric acid (1:1) | | | | | J = 9.0, 6.5 Hz, |
| | | | | | 2H), 6.78 (dd, J = |
| | | | | | 9.7, 2.6 Hz, 1H), |
| | | | | | 6.07 (s, 1H), 3.20 |
| | | | | | (s, 6H). Elemental |
| | | | | | analysis: Anal. |
| | | | | | Cald. for {Compound |
| | | | | | I:nitric acid (1:1)} |
| | | | | | C₁₃H₁₅N₅O₄: |
| | | | | | C, 51.15; H, 4.95; |
| | | | | | N, 22.94%. |
| | | | | | Found: C, 51.36; |
| | | | | | H, 4.93; N, 22.92%. |
| 5- | 10 | Tartaric | Water | B | ¹H NMR (400 |
| (dimethylamino)- | | acid | | | MHz, DMSO-d₆) |
| 2-(pyridin-2- | | | | | δ 12.66 (s, 2H), |
| yldiazenyl)phenol, | | | | | 8.43-8.37 (m, 1H), |
| tartaric acid (1:1) | | | | | 7.87 (td, J = |
| | | | | | 7.8, 7.3, 1.8 Hz, |
| | | | | | 1H), 7.64 (d, J = |
| | | | | | 8.3 Hz, 1H), 7.24- |
| | | | | | 7.16 (m, 2H), |
| | | | | | 6.70 (dd, J = 9.7, |
| | | | | | 2.7 Hz, 1H), 5.76 |
| | | | | | (d, J = 2.6 Hz, |
| | | | | | 1H), 5.05 (s, 2H), |
| | | | | | 4.29 (s, 2H), 3.11 |
| | | | | | (s, 6H). Elemental |
| | | | | | analysis: Anal. |
| | | | | | Cald. for {Compound |
| | | | | | I:tartaric acid |
| | | | | | (1:1)} |
| | | | | | C₁₇H₂₀N₄O₇: |
| | | | | | C, 52.04; H, 5.14; |
| | | | | | N, 14.28%. |
| | | | | | Found: C, 52.23; |
| | | | | | H, 5.15; N, 14.15%. |
| 5- | 11 | Succinic | Acetone | B | ¹H NMR (400 |
| (dimethylamino)- | | acid | | | MHz, DMSO-d₆) |
| 2-(pyridin-2- | | | | | δ 12.11 (s, 1H), |
| yldiazenyl)phenol, | | | | | 8.40 (ddd, J = 4.8, |
| succinic acid | | | | | 1.8, 0.9 Hz, 1H), |
| (1:0.5) | | | | | 7.91-7.82 (m, |
| | | | | | 1H), 7.68-7.61 |
| | | | | | (m, 1H), 7.24- |
| | | | | | 7.15 (m, 2H), |
| | | | | | 6.70 (dd, J = 9.7, |
| | | | | | 2.6 Hz, 1H), 5.76 |
| | | | | | (d, J = 2.6 Hz, |
| | | | | | 1H), 3.11 (s, 6H), |
| | | | | | 2.40 (d, J = |
| | | | | | 0.9 Hz, 2H). |
| | | | | | Elemental |
| | | | | | analysis: Anal. |
| | | | | | Cald. for {Compound |
| | | | | | I:succinic acid |
| | | | | | (1:0.5)} |
| | | | | | C₃₀H₃₄N₈O₆: |
| | | | | | C, 59.79; H, 5.69; |
| | | | | | N, 18.59%. |
| | | | | | Found: C, 59.52; |
| | | | | | H, 5.49; N, 18.68%. |
| 5- | 12 | Ethanedisulfonic | Ethanol | A | ¹H NMR (400 |
| (dimethylamino)- | | acid | | | MHz, DMSO-d₆) |
| 2-(pyridin-2- | | | | | δ 8.54 (d, J = 5.6 |
| yldiazenyl)phenol, | | | | | Hz, 1H), 8.29 (t, |
| ethanedisulfonic | | | | | J = 8.0 Hz, 1H), |
| acid (1:1) | | | | | 8.00 (d, J = 8.5 |
| | | | | | Hz, 1H), 7.52 (dt, |
| | | | | | J = 13.2, 8.0 Hz, |
| | | | | | 2H), 6.81 (dd, J = |
| | | | | | 9.8, 2.6 Hz, 1H), |
| | | | | | 6.16 (s, 1H), 3.23 |
| | | | | | (s, 6H), 2.65 (s, |
| | | | | | 4H). Elemental |
| | | | | | analysis: Anal. |
| | | | | | Cald. for {Compound |
| | | | | | I:ethane-1,2- |
| | | | | | disulfonic acid |
| | | | | | (1:1)} |
| | | | | | C₁₅H₂₀N₄O₇S₂: |
| | | | | | C, 41.94; H, 4.59; |
| | | | | | N, 12.78%. |
| | | | | | Found: C, 41.94; |
| | | | | | H, 4.59; N, 12.78%. |
| 5- | 13 | Oxalic | Ethanol | A | ¹H NMR (400 |
| (dimethylamino)- | | acid | | | MHz, DMSO-d₆) |
| 2-(pyridin-2- | | | | | δ 8.40 (d, J = 4.9 |
| yldiazenyl)phenol, | | | | | Hz, 1H), 7.92- |
| oxalic acid (1:0.5) | | | | | 7.83 (m, 1H), |
| | | | | | 7.65 (d, J = 8.3 |
| | | | | | Hz, 1H), 7.24- |
| | | | | | 7.16 (m, 2H), |
| | | | | | 6.70 (ddd, J = 9.6, |
| | | | | | 2.7, 1.3 Hz, 1H), |
| | | | | | 5.76 (dd, J = 2.7, |
| | | | | | 1.4 Hz, 1H), 3.11 |
| | | | | | (d, J = 1.4 Hz, |
| | | | | | 6H). Elemental |
| | | | | | analysis: Anal. |
| | | | | | Cald. for {Compound |
| | | | | | I:oxalic acid |
| | | | | | (1:0.5)} |
| | | | | | C₂₈H₃₀N₈O₆: |
| | | | | | C, 58.53; H, 5.26; |
| | | | | | N, 19.5%. |
| | | | | | Found: C, 58.84; |
| | | | | | H, 5.05; N, 19.57%. |
| 5- | 14 | Fumaric | Ethanol | B | ¹H NMR (400 |
| (dimethylamino)- | | acid | | | MHz, DMSO-d₆) |
| 2-(pyridin-2- | | | | | δ 13.10 (s, 1H), |
| yldiazenyl)phenol, | | | | | 8.40 (d, J = 4.6 |
| fumaric acid | | | | | Hz, 1H), 7.91- |
| (1:0.5) | | | | | 7.82 (m, 1H), |
| | | | | | 7.64 (dd, J = 8.4, |
| | | | | | 1.5 Hz, 1H), 7.24- |
| | | | | | 7.15 (m, 2H), |
| | | | | | 6.70 (dt, J = 9.7, |
| | | | | | 2.0 Hz, 1H), 6.61 |
| | | | | | (d, J = 1.5 Hz, |
| | | | | | 1H), 5.76 (t, J = |
| | | | | | 2.0 Hz, 1H), 3.11 |
| | | | | | (d, J = 1.5 Hz, |
| | | | | | 6H). Elemental |
| | | | | | analysis: Anal. |
| | | | | | Cald. for {Compound |
| | | | | | I:Fumaric acid |
| | | | | | (1:0.5)} |
| | | | | | C₃₀H₃₂N₈O₆: |
| | | | | | C, 59.99; H, 5.37; |
| | | | | | N, 18.66%. |
| | | | | | Found: C, 59.99; |
| | | | | | H, 5.23; N, 18.53%. |
| 5- | 15 | Ethanesulfonic | Ethyl | A | ¹H NMR (400 |
| (dimethylamino)- | | acid | acetate | | MHz, DMSO-d₆) |
| 2-(pyridin-2- | | | | | δ 8.55 (d, J = 5.7 |
| yldiazenyl)phenol, | | | | | Hz, 1H), 8.31 (t, J = |
| ethanesulfonic | | | | | 8.0 Hz, 1H), |
| acid, monohydrate | | | | | 8.01 (d, J = 8.5 |
| (1:2:1) | | | | | Hz, 1H), 7.54 (dt, |
| | | | | | J = 13.2, 8.0 Hz, |
| | | | | | 2H), 6.81 (dd, J = |
| | | | | | 9.7, 2.6 Hz, 1H), |
| | | | | | 6.20-6.15 (m, |
| | | | | | 1H), 3.23 (d, J = |
| | | | | | 1.5 Hz, 6H), 2.48- |
| | | | | | 2.38 (m, 4H), |
| | | | | | 1.06 (td, J = |
| | | | | | 7.4, 1.4 Hz, 6H). |
| | | | | | Elemental |
| | | | | | analysis: Anal. |
| | | | | | Cald. for {Compound |
| | | | | | I:ethanesulfonic |
| | | | | | acid (1:2)} |
| | | | | | C₁₇H₂₈N₄O₈S₂: |
| | | | | | C, 42.49; H, 5.87; |
| | | | | | N, 11.66%. |
| | | | | | Found: C, 42.8; |
| | | | | | H, 5.69; N, 10.76%. |
| 5- | 16 | Glycolic | Ethanol | A | ¹H NMR (400 |
| (dimethylamino)- | | acid | | | MHz, DMSO-d₆) |
| 2-(pyridin-2- | | | | | δ 8.40 (d, J = 5.0 |
| yldiazenyl)phenol, | | | | | Hz, 1H), 7.87 (tt, |
| glycolic acid | | | | | J = 8.5, 1.5 Hz, |
| (1:0.5) | | | | | 1H), 7.68-7.61 |
| | | | | | (m, 1H), 7.24- |
| | | | | | 7.16 (m, 2H), |
| | | | | | 6.74-6.66 (m, |
| | | | | | 1H), 5.76 (dd, J = |
| | | | | | 2.7, 1.2 Hz, 1H), |
| | | | | | 3.89 (s, 1H), 3.11 |
| | | | | | (d, J = 1.3 |
| | | | | | Hz, 6H). Elemental |
| | | | | | analysis: Anal. |
| | | | | | Cald. for {Compound |
| | | | | | I:glycolic acid |
| | | | | | (1:0.5)} |
| | | | | | C₂₈H₃₂N₈O₅: |
| | | | | | C, 59.99; H, 5.75; |
| | | | | | N, 19.99%. |
| | | | | | Found: C, 60.45; |
| | | | | | H, 5.86; N, 20.20%. |
| 5- | 17 | Para- | Ethanol | A | ¹H NMR (400 |
| (dimethylamino)- | | toluenesulfonic | | | MHz, DMSO-d₆) |
| 2-(pyridin-2- | | acid | | | δ 8.52 (d, J = 5.7 |
| yldiazenyl)phenol, | | | | | Hz, 1H), 8.18 (d, |
| para- | | | | | J = 29.7 Hz, 2H), |
| toluenesulfonic | | | | | 8.00-7.81 (m, |
| acid (1:1) | | | | | 1H), 7.45 (dd, J = |
| | | | | | 8.0, 2.8 Hz, 4H), |
| | | | | | 7.09 (d, J = 7.6 |
| | | | | | Hz, 2H), 6.84- |
| | | | | | 6.71 (m, 1H), |
| | | | | | 6.06 (d, J = 30.9 |
| | | | | | Hz, 1H), 3.20 (dd, |
| | | | | | J = 8.7, 1.8 Hz, |
| | | | | | 6H), 2.27 (s, 3H). |
| | | | | | Elemental |
| | | | | | analysis: Anal. |
| | | | | | Cald. for {Compound |
| | | | | | I:paratoluenesulfonic |
| | | | | | acid (1:1)} |
| | | | | | C₂₀H₂₂N₄O₆: |
| | | | | | C, 57.96; H, 5.35; |
| | | | | | N, 13.52%. |
| | | | | | Found: C, 57.93; |
| | | | | | H, 5.08; N, 13.34%. |
| 5- | 18 | Malic | Ethanol | B | ¹H NMR (400 |
| (dimethylamino)- | | acid | | | MHz, DMSO-d₆) |
| 2-(pyridin-2- | | | | | δ 12.36 (s, 2H), |
| yldiazenyl)phenol, | | | | | 8.43-8.37 (m, |
| malic acid (1:1) | | | | | 1H), 7.87 (ddd, J = |
| | | | | | 9.0, 7.2, 1.8 Hz, |
| | | | | | 1H), 7.64 (d, J = |
| | | | | | 8.2 Hz, 1H), 7.24- |
| | | | | | 7.16 (m, 2H), |
| | | | | | 6.70 (dd, J = 9.7, |
| | | | | | 2.7 Hz, 1H), 5.76 |
| | | | | | (d, J = 2.6 Hz, |
| | | | | | 1H), 5.41 (s, 1H), |
| | | | | | 4.24 (dd, J = 7.8, |
| | | | | | 4.8 Hz, 1H), 3.11 |
| | | | | | (s, 6H), 2.59 (dd, |
| | | | | | J = 15.7, 4.8 Hz, |
| | | | | | 1H), 2.42 (dd, J = |
| | | | | | 15.7, 7.8 Hz, 1H). |
| | | | | | Elemental |
| | | | | | analysis: Anal. |
| | | | | | Cald. for {Compound |
| | | | | | I:malic |
| | | | | | acid (1:1)} |
| | | | | | C₁₇H₂₀N₄O₆: |
| | | | | | C, 54.25; H, 5.36; |
| | | | | | N, 14.89%. |
| | | | | | Found: C, 54.69; |
| | | | | | H, 5.35; N, 14.90% |
| 5- | 19 | Naphthalene- | Ethanol | A | ¹H NMR (400 |
| (dimethylamino)- | | 2-sulfonic | | | MHz, DMSO-d₆) |
| 2-(pyridin-2- | | acid | | | δ 8.55 (d, J = 5.6 |
| yldiazenyl)phenol, | | | | | Hz, 1H), 8.32 (t, |
| naphthalene-2- | | | | | J = 8.0 Hz, 1H), |
| sulfonic acid (1:2) | | | | | 8.12 (s, 2H), 8.02 |
| | | | | | (d, J = 8.6 Hz, |
| | | | | | 1H), 7.99-7.91 |
| | | | | | (m, 2H), 7.91- |
| | | | | | 7.79 (m, 3H), |
| | | | | | 7.69 (dt, J = 8.5, |
| | | | | | 1.5 Hz, 2H), 7.62- |
| | | | | | 7.44 (m, 5H), |
| | | | | | 6.89-6.74 (m, |
| | | | | | 1H), 6.18 (d, J = |
| | | | | | 2.6 Hz, 1H), 3.23 |
| | | | | | (s, 5H). Elemental |
| | | | | | analysis: Anal. |
| | | | | | Cald. for {Compound |
| | | | | | I:naphthalene- |
| | | | | | 2-sulfonic acid |
| | | | | | (1:2)} |
| | | | | | C₃₃H₃₀N₄O₇S₂: |
| | | | | | C, 60.17; H, 4.59; |
| | | | | | N, 8.51%. |
| | | | | | Found: C, 60.11; |
| | | | | | H, 4.34; N, 8.42%. |
| 5- | 20 | Naphthalene- | Ethanol | A | ¹H NMR (400 |
| (dimethylamino)- | | 1,5-disulfonic | | | MHz, DMSO-d₆) |
| 2-(pyridin-2- | | acid | | | δ 8.84 (d, J = |
| yldiazenyl)phenol, | | | | | 8.6 Hz, 2H), 8.54 (d, |
| naphthalene-1,5- | | | | | J = 6.0 Hz, 1H), |
| disulfonic acid, | | | | | 8.36-8.15 (m, |
| monohydrate | | | | | 2H), 8.07-7.84 |
| (1:1:1) | | | | | (m, 3H), 7.62- |
| | | | | | 7.42 (m, 2H), |
| | | | | | 7.39 (ddd, J = 8.9, |
| | | | | | 7.4, 2.0 Hz, 2H), |
| | | | | | 6.86-6.73 (m, |
| | | | | | 1H), 6.14 (d, J = |
| | | | | | 13.8 Hz, 2H), |
| | | | | | 3.31-3.09 (m, |
| | | | | | 6H). Elemental |
| | | | | | analysis: Anal. |
| | | | | | Cald. for {Compound |
| | | | | | I:naphthalene- |
| | | | | | 1,5-disulfonic |
| | | | | | acid (1:1)} |
| | | | | | C₂₃H₂₄N₄O₈S₂: |
| | | | | | C, 50.36; H, 4.41; |
| | | | | | N, 10.21%. |
| | | | | | Found: C, 50.82; |
| | | | | | H, 4.57; N, 9.62%. |
| 5- | 21 | Pyruvic | Acetonitrile | A | ¹H NMR (400 |
| (dimethylamino)- | | acid | | | MHz, DMSO-d₆) |
| 2-(pyridin-2- | | | | | δ 13.81 (s, 1H), |
| yldiazenyl)phenol, | | | | | 8.43-8.36 (m, |
| pyruvic acid (1:1) | | | | | 1H), 7.87 (dddd, J = |
| | | | | | 8.3, 7.3, 1.8, 1.0 |
| | | | | | Hz, 1H), 7.65 (dt, |
| | | | | | J = 8.2, 1.0 Hz, |
| | | | | | 1H), 7.24-7.16 |
| | | | | | (m, 2H), 6.74- |
| | | | | | 6.66 (m, 1H), |
| | | | | | 5.76 (d, J = 2.6 |
| | | | | | Hz, 1H), 3.11 (d, |
| | | | | | J = 1.1 Hz, 6H), |
| | | | | | 2.32 (d, J = 1.1 |
| | | | | | Hz, 3H). |
| | | | | | Elemental |
| | | | | | analysis: Anal. |
| | | | | | Cald. for {Compound |
| | | | | | I:pyruvic |
| | | | | | acid (1:1)} |
| | | | | | C₁₆H₁₈N₄O₄: |
| | | | | | C, 58.62; H, 5.31; |
| | | | | | N, 16.96%. |
| | | | | | Found: C, 58.62; |
| | | | | | H, 5.31; N, 17.00%. |

### Biological Assays

### Example 3

### Cell Lines:

TMPRSS2-ERG fusion positive ERG oncoprotein expressing prostate cancer cell line VCaP, was obtained from the American Tissue Culture Collection (ATCC; Manassas, Va.). The cells were grown in ATCC-recommended cell culture media under cell growth promoting conditions as recommended by the supplier (DMEM media; Gibco, Grand island, NY) supplemented with 10% fetal bovine serum (ATCC; Manassas, Va.). Normal primary endothelial cells, HUVEC-human umbilical vein endothelial cells, were also obtained from ATCC (Endothelial cell media; Cell biologics, Chicago, Ill.) supplemented with 10% FBS) as recommended by the supplier. The passage number of cell lines used in this study ranges from 1 to 6.

### Reagents:

ERG monoclonal antibody (CPDR ERG-MAb; 9FY; Biocare Medical, CA) was developed and characterized at the Center for Prostate Disease Research. RIOK2 mouse monoclonal antibody (TA505140) was from Origene (Rockville, Md.). Antibody against glyceraldehyde phosphate dehydrogenase (GAPDH; sc-25778) was purchased from Santa Cruz Biotechnology (Santa Cruz, Calif.).

### Methods for Immunoblot Assay

The TMPRSS2-ERG fusion positive prostate cancer cell line VCaP was used to study inhibition of ERG expression.

The VCaP cells were seeded in a 10 mm Petri dish (08-772B, Falcon Corning, N.Y.) at a cell density of 2×10⁶ cells per plate. Following 48 hours of incubation at 37° C., cells were exposed with the test compounds (0, 0.001, 0.005, 0.01, 0.05, 0.10, 0.15, 0.2, 0.25, 0.3, 0.35, 0.4 μM) for 48 hours, cells were lysed using Mammalian Protein Extraction Reagent (M-PER: Pierce, Rockford, Il.) containing a protease inhibitor cocktail and phosphatase inhibitor cocktails I& I (Sigma, St Louis, Mo). Cell lysates containing 50 μg of total protein were electrophoresed through 4-12% Bis-Tris Gel (Invitrogen, Carlsbad, Calif.) using running buffer (Nupage MES SDS running buffer, Invitrogen, Carlsbad, Calif.) and the cellular proteins were transferred to PVDF membrane using iBlot (Invitrogen, Carlsbad, Calif.). Membranes were incubated at 4° C. for 12 hours with primary antibodies for ERG, RIOK2 and GAPDH. Following exposure of primary antibodies in 5% blotting grade nonfat milk solution (Bio-Rad, Hercules, Calif.), the membranes were washed with 1× Tris buffered saline containing 0.1% Tween 20 (1×TBST) buffer (Bio-Rad, Hercules Calif.) three times, 5 minutes each at room temperature followed by incubation with secondary antibodies that includes Goat anti-mouse IgG(H+L) secondary antibody (HAF007, Novus biologicals Centennial, CO)) at 1:1000 ratio for ERG, RIOK2 and Goat anti-Rabbit IgG(H+L) secondary antibody, HRP (HAF008, Novus biologicals Centennial, CO) at 1:5000 ratio for GAPDH in 5% blotting grade nonfat milk solution (Bio-Rad, Hercules, Calif.) for 1 hour at 24° C. Finally, the membranes were washed with 1×TBST buffer and developed using the ECL Western blot detection reagent (GE Health Care, Buckinghamshire, UK) by measuring chemiluminescence detected by the iBright system (Invitrogen, Carlsbad, Calif.). The iBright images were analyzed using iBright software to calculate the IC50 of each compound using (GraphPad Prism 7 software).

### Methods for Cell Growth and Inhibition:

The ERG positive VCaP cells were grown as adherent monolayers in 6-well tissue culture dishes (08-772-1B, Falcon Corning, N.Y.), using the appropriate growth medium (DMEM supplemented with 10% FBS) as suggested by the vendor. 48 hours following plating of cells, the appropriate test compound is added to each well of the tissue culture dish at concentrations (0, 0.001, 0.005, 0.01, 0.05, 0.10, 0.15, 0.2, 0.25, 0.3, 0.35, 0.4) μM/per well). The medium was replenished every 24 hours with fresh growth medium containing the same concentration of the same test compound for indicated period of the cell growth inhibition assay for 96 hours. At the end of the treatment the cells were analyzed for viability by labeling for ATPase activity. Cell growth were calculated by measuring bioluminescence using Cell Glow ATPase assay with Cell Titer-Glo luminescent cell viability assay (Promega, Madison, Wis.) detected by the HT Envision System (PerkinElmer, Waltham, Mass.). The average viable cell numbers of each concentration in triplicates were used to calculate the IC₅₀ of each compound with the GraphPad Prism 7 software. Trypan blue dye exclusion method using Countess automated counter (Invitrogen Carlsbad, Calif.) and microscopy photography were used to estimate the fraction of viable cells in each test well.

### Results

The IC₅₀ of ERG protein inhibition (see Immunoblot assay described above) is summarized in Table 2.

TABLE 2

| | IC50 |
|---|---|
| Compound | ERG protein inhibition (μM) |
| (E)-5-(dimethylamino)- | 0.23 |
| 2-(pyridin-2- | |
| yldiazenyl)phenol | |
| (Compound I) | |
| 1 | 0.22 |
| 2 | 0.17 |
| 3 | 0.21 |
| 4 | 0.24 |
| 5 | 0.26 |
| 6 | 0.24 |
| 7 | 0.26 |
| 8 | 0.11 |
| 9 | Undetectable |
| 10 | 0.22 |
| 11 | 0.16 |
| 12 | 0.15 |
| 13 | Undetectable |
| 14 | Undetectable |
| 15 | Undetectable |
| 16 | Undetectable |
| 17 | 0.239 |
| 18 | Undetectable |
| 19 | Undetectable |
| 20 | Undetectable |
| 21 | Undetectable |

IC₅₀ of tested compounds for VCaP cell viability and IC₅₀ of tested compounds for a RIOK2 protein assay (see immunoblot assay described above) are summarized in Table 3.

TABLE 3

| | IC50 | IC50 |
|---|---|---|
| | VCaP Cell Viability | RIOK2 protein |
| Compound | (μM) | inhibition (μM) |
| (E)-5-(dimethylamino)- | 0.13 | 0.25 |
| 2-(pyridin-2- | | |
| yldiazenyl)phenol | | |
| (Compound I) | | |
| 1 | | 0.19 |
| 2 | 0.089 | 0.13 |
| 3 | | 0.20 |
| 4 | | 0.17 |
| 5 | 0.139 | 0.26 |

Compound 2 was also found to be a selective inhibitor of ERG positive cancer cells and did not effect the growth, ERG, and RIOK2 levels of endothelial-derived primary cells. ERG protein is naturally expressed in endothelial cells. HUVEC cells naturally expressing ERG protein did not respond to Compound 2 treatment within the effective concentration range observed in ERG positive VCaP prostate cancer cells (data not shown).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A pharmaceutical composition comprising Compound I: 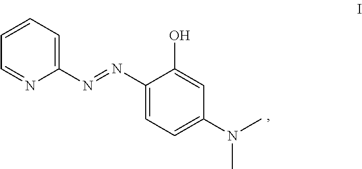 [SMILES: CN(C)c1ccc(/N=N/c2ccccn2)c(O)c1]
wherein at least 97% of Compound I is a methanesulfonic acid salt.

2. The pharmaceutical composition of claim 1, wherein at least 98% of Compound I is a methanesulfonic acid salt.

3. The pharmaceutical composition of claim 1, wherein at least 99% of Compound I is a methanesulfonic acid salt.

4. The pharmaceutical composition of claim 1, further comprising a pharmaceutically acceptable excipient.

5. The pharmaceutical composition of claim 4, wherein at least 98% of Compound I is a methanesulfonic acid salt.

6. The pharmaceutical composition of claim 4, wherein at least 99% of Compound I is a methanesulfonic acid salt.

7. The pharmaceutical composition of claim 4, wherein the pharmaceutical composition is substantially free of solvent.

8. A method of treating a disease associated with overexpression of wild type ERG protein, an altered ERG protein, ERG gene transcription or ERG mRNA translation in a subject in need thereof, comprising administering a therapeutically effective amount of the pharmaceutical composition of claim 1, wherein the disease is prostate cancer, colorectal cancer, Ewing sarcoma, or leukemia.

9. A method of treating ERG-positive prostate cancer, comprising administering a therapeutically effective amount of the pharmaceutical composition of claim 1.

10. The method of claim 8, further comprising administering an additional active agent.

11. A method for preparing a pharmaceutically acceptable salt of Compound I: 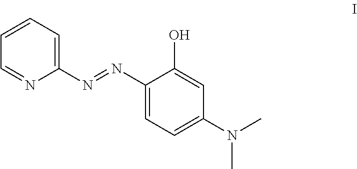 [SMILES: CN(C)c1ccc(/N=N/c2ccccn2)c(O)c1]
wherein the pharmaceutically acceptable salt is dihydroxybenzoic acid salt, hydrochloric acid salt, maleic acid salt, benzenesulfonic acid salt, or methanesulfonic acid salt, comprising:
contacting Compound I with an acid selected from dihydroxybenzoic acid, hydrochloric acid, maleic acid, benzenesulfonic acid, or methanesulfonic acid under conditions sufficient to form the pharmaceutically acceptable salt of Compound I.

12. A method for preparing a pharmaceutically acceptable salt of Compound I: 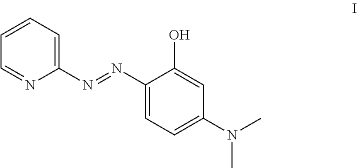 [SMILES: CN(C)c1ccc(/N=N/c2ccccn2)c(O)c1]
wherein the pharmaceutically acceptable salt is dihydroxybenzoic acid salt, hydrochloric acid salt, maleic acid salt, benzenesulfonic acid salt, or methanesulfonic acid salt, comprising:
(a) contacting a Compound A: 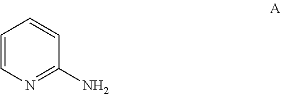
with an oxidizing agent under conditions sufficient to form Compound B: 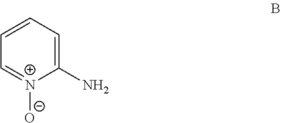
(b) contacting Compound B in the presence of sodium nitrite and a strong acid under conditions sufficient to form a salt of Compound C: 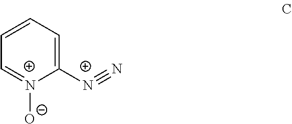
(c) contacting the salt of Compound C with Compound D: 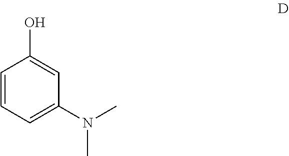
under conditions sufficient to form Compound E: PGP-27,C1,M 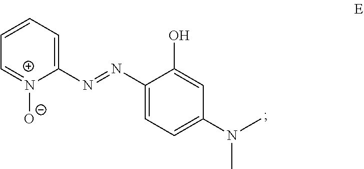 [SMILES: CN(C)c1ccc(/N=N/c2cccc[n+]2[O-])c(O)c1]
(d) contacting Compound E with a molybdenum catalyst and triphenylphosphine under conditions sufficient to form Compound I; and
(e) contacting Compound I with an acid selected from dihydroxybenzoic acid, hydrochloric acid, maleic acid, benzenesulfonic acid, or methanesulfonic acid under conditions sufficient to form the pharmaceutically acceptable salt of Compound I.

13. The method of claim 12, wherein the oxidizing agent is meta-chloroperoxybenzoic acid.

14. The method of claim 12, wherein the strong acid is tetrafluoroboric acid.

15. The method of claim 14, wherein the salt of Compound C is a tetrafluoroboric acid salt.

16. The method of claim 12, wherein the molybdenum catalyst is MoO_{2 .}

17. The method of claim 12, wherein the acid is methanesulfonic acid.

18. The method of claim 17, wherein the pharmaceutically acceptable salt of Compound I is methanesulfonic acid salt.
